SEQUENCE LISTING (1 sequence)

SEQ ID NO: 1 - protein, 10 aa, Homo sapiens
EHWSYGLRPG

(19) United States
(12) Patent Application Publication
(10) Pub. No.: US 2011/0077506 A1
(43) Pub. Date: Mar. 31, 2011

(54) METHODS AND COMPOSITIONS FOR MOLECULAR IMAGING

(76) Inventors: Bastiaan Driehuys, Chapel Hill, NC (US); Rosa Tamara Branca, Durham, NC (US)

(21) Appl. No.: 12/988,463

(22) PCT Filed: Apr. 17, 2009
(86) PCT No.: PCT/US09/40995

§ 371 Date: Dec. 7, 2010

Related U.S. Application Data
(60) Provisional application No. 61/045,809, filed on Apr. 17, 2008.

(51) Int. Cl. A61B 5/055; A61B 6/00

(52) U.S. Cl. 600/420; 600/431

(57) ABSTRACT

Methods for imaging a target cell, tissue, or organ in a subject. In some embodiments, the methods include the steps of (a) administering to the subject a contrast agent that includes a paramagnetic or superparagmanetic material and a targeting moiety that targets the contrast agent to the cell, tissue, or organ; (b) introducing into the target cell, tissue, or organ, or into the vicinity thereof, a hyperpolarized gas; and (c) imaging the target cell, tissue, or organ by detecting the presence of the contrast agent in, on, or near the target cell, tissue, or organ. Also provided are methods for screening for metastasis of a tumor and/or a cancer to the lung of a subject, methods for imaging a cavity in a subject, methods for imaging a target cell, tissue, or organ in a cavity, compositions for performing the disclosed methods, and systems and kits that include the disclosed compositions and/or reagents for performing the disclosed methods.

### CROSS REFERENCE TO RELATED APPLICATION

This application claims the benefit of U.S. Provisional Patent Application Ser. No. 61/045,809, filed Apr. 17, 2008, the disclosure of which is incorporated herein by reference in its entirety.

### GOVERNMENT INTEREST

This presently disclosed subject matter was made with U.S. Government support under Grant No. U24-CA92656-07 awarded by the National Cancer Institute of the National Institutes of Health and Grant Nos. EB 02122 and P41RR005959 awarded by the National Institutes of Health. Thus, the U.S. Government has certain rights in the presently disclosed subject matter.

### TECHNICAL FIELD

The presently disclosed subject matter relates to compositions and methods for imaging targets including, but not limited to cells, tissues, organs, and cavities. The presently disclosed subject matter also relates to methods for screening for metastasis of a tumor and/or a cancer to the lung and/or to a mediastinal lymph node of a subject.

### BACKGROUND

In the United States alone there are over 10 million cancer patients. This year approximately 1.4 million new patients will be diagnosed with cancer and 570,000 will die from the disease (Jemal et al., 2005). While cancer as a confined disease is often treatable, once the disease metastasizes to other organs the outcomes are generally far worse and have not improved over the past 30 years. Metastasis to the lungs is among the most common scenarios. Unfortunately, methods currently do not exist for early detection of lung occult metastases. The problem can, in principle, be solved using sensitive and specific three-dimensional imaging methods (Glasspool & Evans, 2000).

However, imaging of the lung with high resolution and contrast has eluded most modalities, and thus high resolution lung imaging remains an unsolved problem.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides methods for imaging a target cell, tissue, and/or organ in a subject. In some embodiments, the methods comprise (a) administering to the subject a contrast agent comprising a paramagnetic or superparagmanetic material and a targeting moiety that targets the contrast agent to the target cell, tissue, and/or organ; (b) introducing into the target cell, tissue, and/or organ, and/or into the vicinity thereof, a hyperpolarized gas; and (c) imaging the target cell, tissue, and/or organ by detecting the presence of the paramagnetic or superparagmanetic material in and/or on the target cell, tissue, and/or organ. In some embodiments, the paramagnetic or superparagmanetic material comprises a Superparamagnetic Iron Oxide Nanoparticle (SPION). In some embodiments, the target cell, tissue, or organ comprises a cancer cell. In some embodiments, the cancer cell is a metastasized cancer cell. In some embodiments, the metastasized cancer cell is present in the lung, in the mediastinal lymph node, or both the lung and the mediastinal lymph node of the subject.

In some embodiments, the target cell, tissue, and/or organ comprises a target, and the targeting moiety comprises a molecule that binds to the target. In some embodiments, the molecule that binds to the target comprises a peptide, an antibody or a fragment or derivative thereof, or a small molecule. In some embodiments, the antibody or the fragment or derivative thereof comprises a paratope that binds to the target. In some embodiments, the peptide comprises a ligand or receptor that binds to a receptor or ligand present on the surface of the cell or on the surface of a cell present in the tissue or organ. In some embodiments, the ligand comprises a luteinizing hormone releasing hormone (LHRH) peptide or a fragment or derivative thereof that binds to a luteinizing hormone releasing hormone receptor present on the target cell or in a cell present in the target tissue or organ. In some embodiments, the hyperpolarized gas comprises ³He, ¹²⁹Xe, or a combination thereof. In some embodiments, the hyperpolarized gas comprises ¹²⁹Xe. In some embodiments, the imaging comprises a contrast agent-sensing scan with an echo time of from about 3 to about 12 ms and a ventilation-sensing scan with echo time of from about 0.3 to about 1 ms. In some embodiments, the contrast agent-sensing scan has an echo time of about 4 ms. In some embodiments, the ventilation-sensing scan comprises an echo time of about 0.3 ms. In some embodiments, the imaging is based on under-sampled radial imaging. In some embodiments, the imaging is based on Cartesian sampling. In some embodiments, the target cell comprises a cancer cell present in or surrounding a gas-accessible cavity selected from the group consisting of a sinus, the colon, or the uterus of the subject.

The presently disclosed subject matter also provides methods for screening for metastasis of a tumor and/or a cancer to the lung of a subject. In some embodiments, the methods comprise (a) administering to the subject a contrast agent comprising a paramagnetic or superparagmanetic material and a targeting moiety that binds to a tumor and/or a cancer cell; (b) introducing a hyperpolarized gas into the lung of the subject; and (c) imaging the lung by detecting the presence of the paramagnetic or superparagmanetic material bound to the tumor and/or the cancer cell in the lung. In some embodiments, the tumor and/or the cancer comprises a tumor and/or a cancer of the breast, prostate, uterus, or lung. In some embodiments,

The presently disclosed subject matter also provides methods for imaging a target cell, tissue, or organ in a cavity in a subject. In some embodiments, the methods comprise (a) administering to the subject a contrast agent comprising a paramagnetic or superparagmanetic material and a targeting moiety that targets the contrast agent to the cell, tissue, or organ; (b) introducing into the cavity a hyperpolarized gas; and (c) imaging the target cell, tissue, or organ by detecting the presence of the paramagnetic or superparagmanetic material in or on the target cell, tissue, or organ. In some embodiments, the methods further comprise introducing into the cavity a hyperpolarized gas and imaging the cell, tissue, and/or organ before the administration of the contrast agent and imaging the target cell, tissue, and/or organ prior to the administration of the contrast agent. In some embodiments, the paramagnetic or superparagmanetic material comprises a Superparamagnetic Iron Oxide Nanoparticle (SPION). In some embodiments, the target cell, tissue, or organ comprises a cancer cell. In some embodiments, the target cell, tissue, or organ comprises a target, and the targeting moiety comprises a molecule that binds to the target. In some embodiments, the molecule that binds to the target comprises a peptide, an antibody or a fragment or derivative thereof, or a small molecule. In some embodiments, the antibody or the fragment or derivative thereof comprises a paratope that binds to the target. In some embodiments, the peptide comprises a ligand or receptor that binds to a receptor or ligand present on the surface of the cell or on the surface of a cell present in the tissue or organ. In some embodiments, the hyperpolarized gas comprises ³He, ¹²⁹Xe, or a combination thereof. In some embodiments, the hyperpolarized gas comprises ¹²⁹Xe. In some embodiments, the imaging comprises a contrast agent-sensing scan with an echo time of from about 3 to about 8 ms and a ventilation-sensing scan with echo time of from about 0.3 to about 1 ms. In some embodiments, the contrast agent-sensing scan has an echo time of about 4 ms. In some embodiments, the ventilation-sensing scan comprises an echo time of about 0.3 ms. In some embodiments, the imaging is based on under-sampled radial imaging. In some embodiments, the imaging is based on Cartesian sampling. In some embodiments, the cavity selected from the group consisting of a sinus, the colon, or the uterus of the subject.

The presently disclosed subject matter also provides methods for tracking a cell in a subject. In some embodiments, the methods comprise (a) labeling a cell with iron oxide, a paramagnetic or superparamagnetic material, or both, wherein the labeling is in vitro, in vivo, or both; (b) administering the cell to the subject if the cell was labeled in vitro; and (c) serially imaging one or more sites in the subject with hyperpolarized noble gas cell, wherein the imaging is performed before the introduction of the labeled cell into the subject, right after introduction of the labeled cell into the subject, and/or for extended periods after introduction of the labeled cell into the subject whereby the cell is tracked in the subject. In some embodiments, the cell is a stem cell, a T cell, a macrophage, a dendritic cell, or a cancer cell. In some embodiments, the cell is labeled in vivo by introducing into the subject the iron oxide, the paramagnetic or superparamagnetic material, or both in a site in which the iron oxide, the paramagnetic or superparamagnetic material, or both come in contact with the cell to thereby label the cell. In some embodiments, the cell is tracked to a target site selected from the group consisting of a lymph node and a lung.

The presently disclosed subject matter also provides methods for imaging a cavity in a subject. In some embodiments, the methods comprise (a) administering to the subject a contrast agent comprising a paramagnetic or superparagmanetic material and a targeting moiety that targets the contrast agent to the cavity; (b) introducing into the cavity a hyperpolarized gas; and (c) imaging the cavity by detecting the presence of the paramagnetic or superparagmanetic material on the cavity. In some embodiments, the cavity selected from the group consisting of a sinus, the colon, or the uterus of the subject. In some embodiments, the cavity comprises a target molecule disposed therein, and the targeting moiety binds to the target. In some embodiments, the target molecule is present in or on a cell present in the cavity. In some embodiments, the cell present in the cavity is selected from the group consisting of a cancer cell, optionally a metastasized cancer cell; an inflammatory cell, and a macrophage. In some embodiments, the target molecule is a cytokine. In some embodiments, the paramagnetic or superparagmanetic material comprises a Superparamagnetic Iron Oxide Nanoparticle (SPION).

The presently disclosed subject matter also provides magnetic resonance imaging (MRI) contrast agents comprising a paramagnetic or superparagmanetic material and a targeting moiety, wherein the targeting moiety comprises a ligand that binds to a target present in a tissue to be imaged. In some embodiments, the MRI contrast agent is provided in a carrier or diluent that is pharmaceutically acceptable for use in a human. In some embodiments, the contrast agent further comprises a therapeutic agent. In some embodiments, the therapeutic agent is selected from the group consisting of a cytotoxic agent, a chemotherapeutic agent, a radionuclide, an antimetabolite, a suicide gene product, a toxin, a lytic peptide, and combinations thereof.

The presently disclosed subject matter also provides kits comprising an MRI contrast agent as disclosed herein and a noble gas. In some embodiments, the noble gas is hyperpolarized.

The presently disclosed subject matter also provides systems for magnetic resonance imaging (MRI) comprising a contrast agent. In some embodiments, the contrast agent comprises a paramagnetic or superparagmanetic material, and a targeting moiety, wherein the targeting moiety comprises a ligand that binds to a target present in a tissue to be imaged. In some embodiments, the contrast agent is provided in a carrier or diluent that is pharmaceutically acceptable for use in a human. In some embodiments, the presently disclosed systems further comprise a noble gas. In some embodiments, the noble gas is hyperpolarized or alternatively or in addition the system further comprises an apparatus adapted to hyperpolarize the noble gas.

It is an object of the presently disclosed subject matter to provide methods and compositions for imaging cells, tissues, and/or organs in a subject using hyperpolarized gases.

An object of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE DRAWINGS

FIGS. 1A-1C are a series of diagrams illustrating an exemplary approach to the detection of lung metastases in mice bearing human breast and prostate tumor xenografts.

FIG. 1A is a diagram depicting intraperitoneal injection of tagged SPIONs (LHRH-SPIONs) into a mouse prior to imaging. FIG. 1B is a diagram depicting receptor-mediated endocytosis of tagged SPIONs. Cancer cells that express molecules on and/or in their cell membranes that bind to tagged SPIONs (e.g., LHRH-SPIONs) can take up the tagged SPIONs through endocytosis, which can lead to SPIONs accumulating in the cytosolic compartment of the target cell, where they form submicron-sized clusters. FIG. 1C is a diagram showing a mouse mechanically ventilated to deliver hyperpolarized (HP) ³He for imaging. HP ³He transverse magnetization is rapidly dephased by SPIONs that have clustered in the target areas, generating strong magnetic susceptibility gradients. As a result, lung tumors (e.g., primary tumors and/or metastases to the lung) can be readily visualized in magnetic resonance (MR) images as regions of attenuated signal intensity.

FIG. 2 is a series of lung scans (single slices selected from a 3D ³He image) of a tumor-bearing mouse and a control mouse. The top panels show the cancer-sensing scan where signal voids are attributable to Superparamagnetic Iron Oxide Nanoparticles (SPIONs) in cancer cells. The bottom scans are “ventilation sensing” and are used to ensure that signal voids are not the result of ventilation defects.

FIGS. 3A-3F depict the results of ³He-based MRI of a mouse bearing a primary adenocarcinoma with secondary metastases in its lungs. In these longer echo time images (TE=4 ms), a clear signal void is seen in the right lobe. Histological examinations revealed several micrometastases in the same region. Micrometastases were located among healthy lung tissue but the iron accumulation in the micrometastases was able to dephase all the surrounding tissue in the right lobe.

FIG. 3A is a series of 2D hyperpolarized helium images at TE=4 ms from an adenocarcinoma mouse model. The signal loss, clearly visible in the right lobe (circle), represents the detection of the SPION uptake in several micrometastases present in this region. FIG. 3B is an image of hematoxylin & eosin (H&E) staining of the excised lung tissue showing healthy lung parenchyma. FIGS. 3C and 3D are hematoxylin & eosin (H&E) staining images of the right cranial lobe where isolated micrometastases are visible. FIG. 3E show a Prussian blue stained section of the healthy part of the lungs that shows an absence of iron uptake. FIGS. 3F and 3G show a Prussian blue stained section of isolated micrometastases showing iron uptake.

FIGS. 4A and 4B are a series of coronal HP ³He lung MR images formatted with 1 mm slice thickness.

FIG. 4A is a series of HP images (TE=1 ms) from a control (i.e., healthy) showing normal ventilation patterns. FIG. 4B is a series of images from a human prostate mouse model (TE=1 ms) after injection of LHRH-SPIONs. A clear signal defect can be seen in the right cranial lobe (circles).

FIGS. 5A-5C are a series of MR and histological images of a prostate tumor mouse model injected with LHRH-SPION.

FIG. 5A is an image of a 3D volume rendering obtained from the 2D hyperpolarized helium image dataset at TE=1 ms. The signal loss, clearly visible in the right cranial lobe (arrow), is the detection of the SPION uptake in the right cranial mediastinal lymph node. FIG. 5B is an image of hematoxylin & eosin (H&E) staining of the excised lung tissue (1× magnification) including the right cranial mediastinal lymph node. The lung parenchyma appears free of metastases as well as the right cranial mediastinal lymph node. In absence of metastases the functional lymph node will accumulate the iron oxide nanoparticles. The iron accumulation can be seen in helium images of the adjacent lung parenchyma. The dashed lines indicate the lymph node. FIG. 5C is a 4× magnification of the lymph node shown in FIG. 5B. The top panel of FIG. 5C is an H&E stained section, whereas the bottom half of FIG. 5C is a Prussian blue stained section of the same area that reveals iron and indicates uptake of LHRH-SPION by the normally functioning lymph node.

FIGS. 6A-6F are a series of images that show a comparison of the signal loss produced in the short and long echo time ³He images by the iron in the nearby lymph node.

FIG. 6A is an HP ³He lung image from a prostate tumor model mouse at TE=1 ms. FIG. 6B is an image of the same section at TE=4 ms. The signal loss in the right cranial lobe is clearly enhanced at the longer echo time, where it reaches a size of 1.2 mm. FIG. 6C is an H&E stained section of fixed lung tissue from a prostate tumor model mouse. FIG. 6D is a magnification of the signal loss area (TE=4 ms). FIGS. 6E and 6F are magnifications of the same area (20× and 100×, respectively) in the histological slide of FIG. 4C, which reveals a lymph node of about 300 μm.

FIGS. 7A-7D are in vivo axial images of a prostate tumor-bearing mouse. The primary tumor lobe can be easily seen on the upper left corner of each Figure.

FIG. 7A is a spin-echo image. FIG. 7B is a T2 map obtained using a multi-spin-echo sequence: TE=7 ms; TR=4 s; NEchoes=16. The tumor showed lower T2 values in the SPION-targeted area. FIG. 7C shows background-free detection of the outer part of the tumor, highlighting the SPION uptake. FIG. 7D is an H&E stained section of the imaged prostate tumor tissue (1× amplification): the inner necrotic area (lighter area) is surrounded by viable cancer cells (darker area at the periphery). The outer area showed iron positive cells (inset: Prussian blue stained section of the outer area of the tumor; 10× amplification).

### DETAILED DESCRIPTION

### I. General Considerations

Currently, no three-dimensional imaging technique is capable of detecting single lung cancer cells or even very small numbers of cancer cells in the lung. The most common imaging method, x-ray computed tomography (CT), is capable of detecting masses as small as 1 mm³, but this typically represents more than 1 million cells. Additionally, CT is non-specific, meaning that it cannot distinguish between benign and malignant masses because its sole contrast mechanism is based on tissue density.

More particularly, CT is unable to reliably distinguish between metastases and benign lesions such as granulomas and pulmonary lymphoid nodules. Specifically, CT can generate false-positive results caused by hamartomas, granulomas (resulting from tuberculosis, histoplasmosis, and Wegener granulomatosis), sarcoidosis, silicosis, small infarcts, small areas of fibrosis, and intrapulmonary lymph nodes (Kronawitter et al., 1999; Silvestri et al., 2003).

Therefore, despite its considerable anatomical resolution, the lack of molecular information derived from CT often results in an unsatisfactory clinical assessment (Margaritora et al., 2002; Kayton et al., 2006; Marom & Chasen, 2006) and creates a clinical management dilemma. In such cases, management options include surgical resection, transthoracic needle biopsy, or monitoring with serial chest radiographs. Of these options, monitoring is most commonly adopted for small lesions (<5 mm). Although this choice avoids potentially unnecessary surgery or biopsy risks, it is clearly detrimental in cases when lesions are later found to be malignant.

Knowledge of whether metastases are present is also important for guiding decisions about resecting a patient's primary tumor. The presence of lung metastases increases the potential risk of local recurrence after resection of the primary tumor.

CT also exposes the patient to ionizing radiation. Thus, CT lacks both sensitivity and specificity for early cancer detection and the modality must be used sparingly due to concerns about radiation dose.

A more powerful solution would be one based on “molecular imaging”, in which an image is generated that reflects an upregulation of a receptor, protein, or cell type of interest. Molecular imaging can be achieved by a variety of modalities, with the most prominent being the nuclear imaging techniques of Positron Emission Tomography (PET) and Single Photon Emission Computed Tomography (SPECT). These modalities are based on injection of radioactive isotopes that have been linked to certain targeting moieties that then seek out and bind to receptors or cells of interest. PET is based on positron emission and SPECT on gamma ray emission. However, while PET and SPECT have high sensitivity to small numbers of probe molecules, they are both characterized by poor spatial resolution. More importantly, these modalities typically generate very large background signals from non-specific emission or scattering of the gamma rays that form the basis of detection (Rohren et al., 2004). These factors limit the nuclear methods in their abilities to detect small numbers of cells.

Molecular imaging is also possible using magnetic resonance imaging (MRI) and this can have considerable advantages. Notably, MRI does not employ ionizing radiation, and additionally provides the ability to overlay high-resolution anatomical images on the usually lower-resolution molecular image. Molecular imaging using MRI can employ dedicated contrast agents that bind to the targets of interest and then alter the MRI contrast in the vicinity.

As disclosed herein, agents that are employable in molecular MRI strategies include a paramagnetic or superparamagnetic material. Exemplary paramagnetic or superparamagnetic materials include magnetism-engineered nanoparticles such as, but not limited to Superparamagnetic Iron Oxide Nanoparticles (SPIONs; see Wang et al., 2001), gadolinium complexes, dysprosium complexes, monocrystalline iron oxide nanoparticles (MIONs; Muldoon et al., 1995), Ultrasmall Super-paramagnetic Iron Oxide Particles (USPIOs; Reimer et al., 1990), cobalt-based nanoparticles and nanotubes, and other iron oxide-based nanoparticles such as, but not limited to magnetic engineered iron oxide (MEIO) and metal-doped MEIOs (e.g., Mn-MEIO, Co-MEIO, and Ni-MEIO). These particles can be very small (in some embodiments about 5 nm), and can therefore freely diffuse to targets of interest. Furthermore, their composition endows them with a very strong attenuating effect on the MRI signal in their vicinity because they strongly distort the local magnetic field (reduced T₂*). Furthermore, paramagnetic or superparamagnetic materials (e.g., SPIONs) can be linked to peptide sequences (Berry & Curtis, 2003) that allow them to specifically target certain cells or receptors.

One organ where targeted MRI imaging has as yet remained unsatisfactory is the lung. One reason for the failure of current targeted paramagnetic or superparamagnetic materials (e.g., SPIONs) imaging strategies to successfully image the lung is that the lung is generally devoid of water (the normal signal source in MRI). Additionally, typical contrast mechanisms used to detect accumulation of paramagnetic or superparamagnetic materials (e.g., SPIONs) are not possible in the lung. SPION contrast, for example, results from a reduction of T₂* (transverse relaxation), and therefore SPIONs and other paramagnetic or superparamagnetic materials can show up as dark spots in a T₂*-weighted MRI image. Unfortunately, T₂* in the lungs is exceedingly short under normal conditions, which renders lung tissue nearly invisible even in the absence of SPIONs or other paramagnetic or superparamagnetic materials. Therefore, conventional MRI cannot be used to detect SPIONs in the lung, and thus SPIONs and other paramagnetic or superparamagnetic materials have not been successfully implemented as a strategy for imaging the lungs (e.g., for detecting lung cancer or lung metastases).

Targeted paramagnetic or superparamagnetic materials (e.g., SPIONs) can accumulate in cancer cells in the lung. Such paramagnetic or superparamagnetic materials can be targeted to cancer cells by complexing them with, for example, luteinizing hormone releasing hormone (LHRH; Glu-His-Trp-Ser-Tyr-Gly-Leu-Arg-Pro-Gly; SEQ ID NO: 1), the receptors of which are over-expressed in some cancer cells (Leuschner, 2006). In histology experiments, SPIONs accumulate in primary and metastatic tumors in linear proportion to the number of cancer cells present (Leuschner et al., 2006). The SPIONs are taken up by the cancer cells in the lung through endocytosis and accumulate up to 452 picogram/cell. Moreover, LHRH receptors are not present in healthy lung tissue and therefore paramagnetic or superparamagnetic materials (e.g., SPIONs) do not accumulate in the lung unless cancer cells are present.

Thus, if methods could be found to image the accumulation of such paramagnetic or superparamagnetic materials (e.g., SPIONs) in lung cancer cells, these would represent significant advances in early detection of lung cancer metastases. Disclosed herein are methods for imaging cells, tissues, organs, and other sites that are difficult to image using standard MRI techniques.

### II. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used herein, including the claims, unless the context in which the term appears clearly indicates otherwise. Thus, for example, the phrases “a cell” and “the cell” can refer to one or more cells and can include, but are not limited to cells, tissues, and/or organs.

As used herein, the term “or” is used in the “inclusive” sense of “and/or” and not the “exclusive” sense of “either/or”, unless specifically indicated otherwise.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to employ the disclosed methods and compositions.

As used herein, the phrase “small molecule” refers to a compound, for example an organic compound, with a molecular weight of in some embodiments less than about 10,000 daltons, in some embodiments less than about 5,000 daltons, in some embodiments less than about 1,000 daltons, in some embodiments less than about 750 daltons, in some embodiments less than about 600 daltons, and in some embodiments less than about 500 daltons. A small molecule also can have a computed log octanol-water partition coefficient in some embodiments in the range of about −4 to about +14, and in some embodiments in the range of about −2 to about +7.5.

The term “ligand” as used herein refers to a molecule or other chemical entity having a capacity for binding to a target. A ligand can comprise a peptide, an oligomer, a nucleic acid (e.g., an aptamer), a small molecule (e.g., a chemical compound), an antibody or a fragment or derivative thereof, a nucleic acid-protein fusion, and/or any other affinity agent. In some embodiments, a ligand can comprise a targeting moiety.

The term “targeting”, as used herein to describe the in vivo activity of a ligand following administration to a subject, refers to the preferential movement and/or accumulation of a ligand in a target cell, tissue, organ, or other location as compared to a control cell, tissue, or organ.

The terms “selective targeting” of “selective homing” as used herein each refer to a preferential localization of a ligand that results in an amount of ligand in a target cell, tissue, organ, or other location that is in some embodiments about 2-fold greater than an amount of ligand in a control cell, tissue, organ, or other location (i.e., a non-targeted cell, tissue, organ, or other location), in some embodiments about 5-fold or greater, and in some embodiments about 10-fold or greater. The terms “selective targeting” and “selective homing” also refer to binding or accumulation of a ligand in a target cell, tissue, organ, or other location concomitant with an absence of targeting to a control cell, tissue, organ, or other location, in some embodiments the absence of targeting to all control cells, tissues, organs, and other locations.

The term “absence of targeting” is used herein to describe substantially no binding or accumulation of a ligand in one, some, or all control cells, tissues, organs, or other locations where an amount of ligand would be expected to be detectable, if present.

The terms “targeting ligand”, “targeting molecule”, and “targeting moiety”, as used herein each refer to a ligand that displays targeting activity. In some embodiments, a targeting ligand displays selective targeting. In some embodiments, a targeting ligand comprises a peptide that binds to a receptor present on the surface of a target cell, tissue, or organ.

The term “binding” refers to an affinity between two molecules, for example, a ligand and a target molecule. As used herein, the term “binding” refers to a specific binding of one molecule for another in a mixture of molecules. In some embodiments, the binding of a ligand to a target molecule can be considered specific if the binding affinity is about 1×10⁴ M⁻¹ to about 1×10⁶ M⁻¹ or greater.

The term “tumor” as used herein refers to both primary and metastasized solid tumors and carcinomas of any tissue in a subject, including but not limited to breast; colon; rectum; lung; oropharynx; hypopharynx; esophagus; stomach; pancreas; liver; gallbladder; bile ducts; small intestine; urinary tract including kidney, bladder and urothelium; female genital tract including cervix, uterus, ovaries (e.g., choriocarcinoma and gestational trophoblastic disease); male genital tract including prostate, seminal vesicles, testes and germ cell tumors; endocrine glands including thyroid, adrenal, and pituitary; skin (e.g., hemangiomas and melanomas), bone or soft tissues; blood vessels (e.g., Kaposi's sarcoma); brain, nerves, eyes, head and neck (e.g. head and neck squamous cell carcinomas; HNSCC) and meninges (e.g., astrocytomas, gliomas, glioblastomas, retinoblastomas, neuromas, neuroblastomas, Schwannomas and meningiomas). The term “tumor” also encompasses solid tumors arising from hematopoietic malignancies such as leukemias, including chloromas, plasmacytomas, plaques and tumors of mycosis fungoides and cutaneous T-cell lymphoma/leukemia, and lymphomas including both Hodgkin's and non-Hodgkin's lymphomas. Non-limiting examples of tumors and/or cancers that are known to metastasize to the lungs include tumors and/or cancers of the breast, colon, prostate, stomach, ovary, kidney, esophagus, testis, head and neck cancers, choriocarcinomas, and malignant melanoma.

The term “subject” as used herein refers to any invertebrate or vertebrate species. The methods of the presently disclosed subject matter are particularly useful in the treatment and diagnosis of warm-blooded vertebrates. Thus, the presently disclosed subject matter concerns mammals and birds. More particularly provided is the treatment and/or diagnosis of mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses, or mammals that are clinically relevant model species, such as mice and rats. Also provided is the treatment of birds, including the treatment of those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, provided is the treatment of livestock, including, but not limited to, domesticated swine (pigs and hogs), ruminants, horses, poultry, and the like.

III. MRI with Hyperpolarized Noble Gases

Polarized gases can be collected, frozen, thawed, and used in MRI and NMR spectroscopy applications. For ease of description, the term “frozen polarized gas” refers to a polarized gas that has been frozen into a solid state. The term “liquid polarized gas” refers to a polarized gas that has been or is being liquefied into a liquid state. Although each term includes the word “gas”, this word is used to name and descriptively track the gas that is produced via a hyperpolarizer to obtain a polarized “gas” product. Thus, as used herein, the term “gas” is used in certain instances to descriptively indicate a hyperpolarized noble gas product and can be used with modifiers such as solid, frozen, dissolved, and liquid to describe the state or phase of that product. Also, in some embodiments, the hyperpolarized gas is processed such that it is a pharmaceutical grade product suitable for in vivo delivery to a subject (e.g., a human subject). For example, in some embodiments, the ¹²⁹Xe gas product is formulated to have less than about 10 ppb (parts per billion) alkali metal therein, and in some embodiments has less than about 1 ppb alkali metal therein.

Various techniques can be employed to accumulate and capture polarized gases. For example, U.S. Pat. No. 5,642,625 to Cates et al. describes a high volume hyperpolarizer for spin-polarized noble gas, and U.S. Pat. Nos. 5,809,801 and 5,860,295 to Cates et al. describes a cryogenic accumulator for spin-polarized ¹²⁹Xe. U.S. Pat. No. 6,079,213 to Driehuys et al., entitled “Methods of Collecting, Thawing, and Extending the Useful Life of Polarized Gases and Associated Apparatus”, describes an improved accumulator and collection and thaw methods. The entire disclosures of these documents are hereby incorporated by reference.

As used herein, the terms “hyperpolarize”, “polarize”, and the like are used interchangeably and refer to artificially enhancing the polarization of certain noble gas nuclei over the natural or equilibrium levels. Such an increase can be desirable because it can allow stronger imaging signals corresponding to better MRI images and spectroscopy signals of the gas in the body. By way of example, hyperpolarization can be induced by spin-exchange with an optically pumped alkali-metal vapor or alternatively by metastability exchange. See U.S. Pat. No. 5,545,396 to Albert et al. See also U.S. Pat. Nos. 5,789,921; 5,785,953; 6,123,919; 6,593,144; and 6,241,966.

Additional U.S. Patents that relate to the generation, storage, and use of hyperpolarized gases include U.S. Pat. Nos. 5,612,103; 6,128,918; 6,199,385; 6,237,363; 6,284,222; 6,295,834; 6,305,190; 6,346,229; 6,427,452; 6,430,960; 6,484,532; 6,488,910; 6,491,895; 6,526,778; 6,537,825; 6,599,497; 6,630,126; 6,667,008; 6,696,040; and 6,991,777, the disclosure of each of which is incorporated herein by reference in its entirety.

### IV. Methods of Imaging a Target

As disclosed herein, the historical challenges of imaging the lung can be overcome using the presently disclosed MRI technology based on hyperpolarized (HP) gases. These gases, which include the stable isotopes ³He, ¹²⁹Xe, ²¹Ne, and ⁸³Kr, are amenable to having their signals enhanced by a factor of, in some embodiments, 100,000 or more by an off-line optical process that aligns their nuclei. This extraordinary signal enhancement makes it possible to image these gases at high resolution after they have been administered to the subject, such as by being inhaled into the lungs.

This capability is changing the face of pulmonary imaging by permitting high-resolution MR images of lung function to be made for the first time. Because these gases have a higher mobility in the lungs, they have a longer transverse relaxation time (T₂*) than the protons in lung tissue. Because of the T₂* of HP gases is normally long, there is plenty of dynamic range for shortening the T₂* by the accumulation of paramagnetic or superparamagnetic materials (e.g., SPIONs). Thus, for the first time, the problem of detecting paramagnetic or superparamagnetic materials (e.g., SPIONs) in the lung (e.g., in lung cancer cells and/or in tumor and/or cancer cells that have metastasized to the lung) can be directly addressed by using HP noble gas (e.g., ³He or ¹²⁹Xe) MRI as a “read-out”.

Thus, in some embodiments the presently disclosed subject matter relates to using HP gas MRI to reveal the accumulation of targeted paramagnetic or superparamagnetic materials (e.g., SPIONs) in the lung. In some embodiments, the presently disclosed subject matter also relates to the image acquisition and analysis strategies needed to do so. In some embodiments, the presently disclosed subject matter relates to the use of HP gas to image cancer in other cavities, such as the colon, a sinus, or the uterus in a subject.

Thus, in some embodiments the presently disclosed subject matter provides methods for imaging a target cell, tissue, and/or organ in a subject. In some embodiments, the methods comprise (a) administering to the subject a contrast agent comprising a paramagnetic or superparamagnetic material and a targeting moiety that targets the contrast agent to the cell, tissue, or organ; (b) introducing into the target cell, tissue, or organ, or into the vicinity thereof, a hyperpolarized gas; and (c) imaging the target cell, tissue, or organ by detecting the presence of the contrast agent in or on the target cell, tissue, or organ. In some embodiments, the contrast agent comprises a SPION.

In some embodiments, a pre-contrast scan (also referred to herein as “pre-imaging”) is performed, in which the target cell, tissue, and/or organ in a subject is imaged after administering the hyperpolarized gas but before administering the contrast agent (or, alternatively, before the contrast agent accumulates in, on, or near the target cell, tissue, or organ). The pre-contrast scan can then be compared to the image generated in the same tissue in the presence of the contrast agent (also referred to herein as a “post-contrast scan”), and differences in signal intensities can be identified.

In some embodiments, the presence of the contrast agent is detected by detecting a signal void that results from the presence of the contrast agent.

In some embodiments, detection of a contrast agent is accomplished not by detecting a signal void that results form the presence of the contrast agent (i.e., negative contrast), but with positive contrast. Exemplary techniques for detecting paramagnetic or superparamagnetic materials with positive contrast include, but are not limited to selective excitation (Felfoul et al., 2008), off resonance saturation (Zurkiya & Hu, 2006), intermolecular Multiple Quantum Coherences (Branca et al., 2009), susceptibility weighted echo time encoding (Kim et al., 2007), partially refocused sequences (Seppenwoolde et al., 2007), Sweep Imaging with Fourier Transformation (SWIFT), Ultra short echo time detection, and indirect detection (Ward et al., 2000).

As used herein, the phrase “target cell, tissue, and/or organ” refers to a cell, a tissue, and/or an organ the imaging of which is desirable. In some embodiments, the imaging comprises MRI imaging, and in some embodiments the MRI imaging is facilitated by the presence in, on, and/or near the cell, tissue, or organ of one or more hyperpolarized gases and a contrast agent. Target cells, tissues, and/or organs include, but are not limited to a cancer cell (e.g., a primary tumor cell or a metastasized cell), a macrophage (e.g., a lung macrophage or a macrophage present within a lymph node), a lymph node (e.g., a mediastinal lymph node), and a lung.

IV.A. Contrast Agents

In some embodiments the presently disclosed subject matter provides methods for imaging a target cell, tissue, or organ in a subject. In some embodiments, the methods comprise (a) administering to the subject a contrast agent comprising a paramagnetic or superparamagnetic materials and a targeting moiety that targets the contrast agent to the cell, tissue, or organ; (b) introducing into the target cell, tissue, or organ, or into the vicinity thereof, a hyperpolarized gas; and (c) imaging the target cell, tissue, or organ by detecting the presence of the contrast agent in or on the target cell, tissue, or organ.

As such, in some embodiments a contrast agent comprises a SPION. As used herein, the term “SPION” refers to a Superparamagnetic Iron Oxide Nanoparticle such as, but not limited to the SPIONs disclosed in U.S. Patent Application Publication No. 20060140871 and in Leuschner et al., 2006, the entirety of each of which is incorporated herein by reference. SPIONs can offer significant advantages over other potential contrast agents in that they have very large effects, which are propagated over long distances, as a result of their extremely large induced magnetic moments. With respect to MRI, the surrounding MRI-detectable nuclei (e.g., water molecules for traditional MRI, atoms of hyperpolarized gas with respect to some embodiments of the presently disclosed subject matter) can act as signal amplifiers and detectors for the magnetic field gradients induced by the nanoparticles. SPIONs also exhibit improved pharmacokinetics relative to other contrast agents. Administered intravenously and/or intraperitoneally, SPIONs slowly extravasate from the vascular into the interstitial space, where they travel through the interstitial-lymphatic fluid to target small nodal metastases (see Harisinghani et al., 2003).

SPIONs and other paramagnetic or superparamagnetic materials can be functionalized to include targeting agents that target the SPIONs or the paramagnetic or superparamagnetic materials for accumulation in target cells, tissues, and/or organs of choice. For example, paramagnetic or superparamagnetic materials (e.g., SPIONs) can be functionalized with cyanogen bromide (see Liu et al., 2007, citing Marshall & Rabinowitz, 1976) or carbodiimide reactions (see Rubio et al., 2001), and can thereafter be conjugated to peptides, antibodies and fragments and derivatives thereof, and small molecules, among others, to target cells, tissues, and/or organs of choice using well known techniques.

In some embodiments, the presently disclosed subject matter relates to contrast agents that comprise a targeting moiety. Any targeting moiety can be employed in the compositions and methods of the presently disclosed subject matter, provided that the targeting moiety binds to a cell, tissue, or organ of interest (i.e., a target) in order to facilitate accumulation of the contrast agent in, on, or near the cell, tissue, or organ of interest (e.g., a target cell, tissue, or organ, and/or a cavity adjacent to or containing the target cell, tissue, or organ). In some embodiments, a targeting moiety comprises luteinizing hormone releasing hormone (LHRH; Glu-His-Trp-Ser-Tyr-Gly-Leu-Arg-Pro-Gly; SEQ ID NO: 1), which binds to tumor cells and other targets that express the LHRH receptor (see Leuschner et al., 2006). For example, certain adenocarcinomas have been shown to express the LHRH receptor, and metastases of adenocarcinomas to the lung can be imaged using LHRH-conjugated paramagnetic or superparamagnetic materials (e.g., LHRH-SPIONs). It is recognized, however, that the use of LHRH-conjugated paramagnetic or superparamagnetic materials is merely exemplary, and that other targeting moieties can be employed with the methods of the presently disclosed subject matter. For example, a targeting moiety can be designed that interacts with one or more known tumor-associated antigens if the target to be imaged is a tumor and/or a cancer cell. Many tumor-associated antigens have been characterized, and any of these can be employed as targets.

In some embodiments, two echo times and two scans are employed, although the use of two echo times is not necessary. However, if one does a long echo time scan (Le., a “contrast agent-sensing scan”) and sees signal voids, the question can arise as to whether this is attributable to ventilation defects or the presence of the contrast agent. For this reason, in some embodiments a “ventilation-sensing scan” is also performed with a shorter echo time to determine if adequate ventilation with the hyperpolarized gas has occurred. It is noted that as familiarity with imaging different target sites increases, the voids that result from the presence of the contrast agent can be distinguished from those resulting from ventilation defects. In some embodiments, the two separate scans are performed in a single breath-hold, while in some embodiments the two separate scans are performed in two separate breath-holds.

In some embodiments, the contrast agent further comprises a therapeutic agent. Therapeutic agents that are useful for cancer therapy, for example, include but are not limited to cytotoxic agents, chemotherapeutic agents, radionuclides, antimetabolites, suicide gene products, toxins, lytic peptides, and combinations thereof. See e.g., Walther & Stein, 1999; and references cited therein. Studies using ligand/drug conjugates have demonstrated that a chemotherapeutic agent can be linked to a ligand to produce a conjugate that maintains the binding specificity of the ligand and the therapeutic function of the agent. For example, doxorubicin has been linked to antibodies or peptides and the ligand/doxorubicin conjugates display cytotoxic activity (Shih et al., 1994; Lau et al., 1995; Sivam et al., 1995; PCT International Publication No. WO 98/10795). Similarly, other anthracyclines, including idarubicin and daunorubocin, have been chemically conjugated to antibodies, which have facilitated delivery of effective doses of the agents to tumors (Aboud-Pirak et al., 1989; Rowland et al., 1993). Other chemotherapeutic agents include cis-platinum (Schechter et al., 1991), methotrexate (Shawler et al., 1988; Fitzpatrick & Garnett, 1995) and mitomycin-C (Dillman et al., 1989). Similar conjugation techniques can be employed to conjugate these molecules and other therapeutic agents to the contrast agents of the presently disclosed subject matter in order to produce a contrast agent that can be used both to image a cell, tissue, and/or organ of interest as well as to deliver the therapeutic agent to the cell, tissue, and/or organ of interest.

In some embodiments of the presently disclosed subject matter, a therapeutic agent comprises a radionuclide. Representative radionuclides include, but are not limited to ¹³¹I and ^{99m}Tc. Additional therapeutic agents that can be conjugated to the contrast agents disclosed herein and used in accordance with the methods and compositions of the presently disclosed subject matter include, but are not limited to alkylating agents such as melphalan and chlorambucil; vinca alkaloids such as vindesine and vinblastine; antimetabolites such as 5-fluorouracil, 5-fluorouridine, and derivatives thereof; toxins such as Pseudomonas exotoxin, diphtheria toxin, and ricin (see e.g., FitzGerald & Pastan, 1989); and hecate and related lytic peptides (see e.g., U.S. Pat. Nos. 5,773,413; 5,861,478; 6,875,744; and 7,381,704); as well as combinations thereof.

IV.B. Routes of Administration

The presently disclosed imaging compositions can be administered to a subject in any form and/or by any route(s) of administration. In some embodiments, the contrast agent is selected from the group including but not limited to an oral formulation, a peroral formulation, a buccal formulation, an enteral formulation, a pulmonary formulation, an inhalable formulation, a rectal formulation, a vaginal formulation, a nasal formulation, a lingual formulation, a sublingual formulation, an intravenous formulation, an intraarterial formulation, an intracardial formulation, an intramuscular formulation, an intraperitoneal formulation, an intratumoral formulation, an intracranial formulation, an intracutaneous formulation, a subcutaneous formulation, an aerosolized formulation, an ocular formulation, an implantable formulation, a depot injection formulation, and combinations thereof. In some embodiments, the route of administration is selected from the group including but not limited to oral, peroral, buccal, enteral, pulmonary, inhalation, rectal, vaginal, nasal, lingual, sublingual, intravenous, intraarterial, intracardial, intramuscular, intraperitoneal, intracranial, intracutaneous, intratumoral, subcutaneous, ocular, via an implant, via a depot injection, and combinations thereof. Where applicable, continuous infusion can enhance accumulation of an imaging composition at a target site (see e.g., U.S. Pat. No. 6,180,082). In some embodiments, the contrast agents of the presently disclosed subject matter are administered intraperitoneally, and in some embodiments the contrast agents of the presently disclosed subject matter are administered intravenously. It is understood that the formulations and routes of administration are not mutually exclusive, and that combinations of any or all formulations and routes of administration can be employed with the methods and compositions of the presently disclosed subject matter.

The contrast agents of the presently disclosed subject matter can be administered to the subject at any time sufficient for allowing the contrast agents to accumulate and be present in, on, or in the vicinity of the target cell, tissue, or organ when the imaging takes place. Such a time can be impacted both by the rate at which the contrast agent reaches the target cell, tissue, or organ as well as the rate at which the contrast agent is cleared from the target cell, tissue, or organ. As such, a contrast agent can be administered to the subject in some embodiments about 1 hour before imaging, in some embodiments about 3 hours before imaging, in some embodiments about 12 hours before imaging, and in some embodiments about 24 hours before imaging.

In some embodiments, the hyperpolarized gas is introduced into the subject by inhalation. For imaging targets at sites other than the lungs, the hyperpolarized gas can be introduced by other standard instillation techniques.

### IV.C. Formulations

An imaging composition as described herein comprises in some embodiments a composition that includes a pharmaceutically acceptable carrier. Suitable formulations include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

The contrast agents can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt).

The contrast agents can also be formulated in rectal compositions (e.g., suppositories or retention enemas containing conventional suppository bases such as cocoa butter or other glycerides), creams, or lotions.

The contrast agents can also be formulated in inhalable compositions.

In some embodiments, the presently disclosed subject matter employs an imaging composition that is pharmaceutically acceptable for use in humans. One of ordinary skill in the art understands the nature of those components that can be present in an imaging composition that is pharmaceutically acceptable for use in humans and also what components should be excluded from an imaging composition that is pharmaceutically acceptable for use in humans.

### IV.D. Doses

The term “effective amount” is used herein to refer to an amount of an imaging composition (e.g., a composition comprising a contrast agent) sufficient to produce a visible image. Actual dosage levels of an imaging composition of the presently disclosed subject matter can be varied so as to administer an amount of the contrast agent that is effective to achieve the desired imaging for a particular subject and/or application. The selected dosage level can depend upon a variety of factors including the affinity and/or avidity of the contrast agent for the biomolecule to which it is intended to bind, the formulation, the route of administration, and the abundances of the biomolecule in the target tissue and the surrounding tissue in the subject being imaged. In some embodiments, a minimal dose is administered, and dose is escalated in the absence of dose-limiting toxicity to a minimally effective amount. Determination and adjustment of an effective dose, as well as evaluation of when and how to make such adjustments, are known to those of ordinary skill in the art of medicine.

For administration of an imaging composition as disclosed herein, conventional methods of extrapolating human dosage based on doses administered to a murine animal model can be carried out using techniques known to one of ordinary skill in the art. Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al., 1966. Briefly, to express a mg/kg dose in any given species as the equivalent mg/m² dose, multiply the dose by the appropriate km factor. In an adult human, 100 mg/kg is equivalent to 100 mg/kg×37 kg/m²=3700 mg/m².

With respect to hyperpolarized gas, the dose can be adjusted to give adequate imaging signal in view of the subject to be imaged. As such, the dose for the hyperpolarized gas(es) can be in some embodiments about 0.5 to 2 liters for a human subject. For animal subjects, the doses can be administered over many breaths at a volume of about 1 ml/100 mg of body weight. For example, a typical mouse can consume about 100 ml of gas, while a typical a rat can consume about 1000 ml.

### V. Other Applications

Given that a paramagnetic or superparamagnetic material (e.g., a SPION) influences an MRI signal based at least in part on the its magnetic character, it is also possible to extend the instant disclosure by taking advantage of condition under which a localized accumulation of iron occurs. For example, a hyperpolarized gas can be employed to image an embolism (e.g., a pulmonary embolism) using the embolism itself as the contrast agent.

Additionally, it has been determined that the mediastinal lymph nodes adjacent to the pulmonary regions in a subject can take up targeted and untargeted iron oxide. The accumulation of the iron oxide in the mediastinal lymph nodes has an effect on HP noble gas MRI images, and thus detecting untargeted iron oxides by HP noble gas MRI is a sensitive method for mediastinal lymph node staging of cancer, among other uses. Currently, untargeted iron oxides are used in conjunction with conventional proton MRI, but the results have been unsatisfactory. Although not wishing to be bound by any particular theory of operation, it appears that macrophages present in normal lymph nodes phagocytose the iron oxide particles, leading to a decrease in signal intensity in MRI. Metastatic lymph nodes that lack macrophages, on the other hand, do not take up the contrast agent, and thus no change in signal intensity is seen on images after contrast enhancement.

Another application of the presently disclosed subject matter is the tracking of magnetically-labeled cells in a subject (e.g., in the lung or in another similarly difficult to image space in the subject). Cells that can be tracked in a subject include, but are not limited to stem cells and/or cells of the immune system. Representative cells of the immune system include, but are not limited to lymphocytes (e.g., T cells), macrophages, and dendritic cells.

For example, isolated cells (e.g., stem cells, T cells, macrophages, dendritic cells, or other type of cells) can be labeled in vitro with superparamagnetic iron-oxide particles, with other paramagnetic or superparamagnetic compounds, and/or with iron oxide. The in vitro labeled cells are then introduced into the subject at a site of interest (e.g., by local injection) and/or at a site that would be expected to introduce the labeled cells into the bloodstream and/or lymphatic system of the subject (e.g., by i.v. injection, i.p. injection, etc.) In some embodiments, the subject (e.g., a mammal such as a mouse or a human) has a natural or experimentally-induced local inflammation in a tissue or organ of interest (e.g., the lung). A non-limiting experimentally induced local inflammation includes an asthma model (e.g., a mouse asthma model).

To track cells of interest in the subject, serial images of the subject can be generated with HP noble gas before the introduction of the labeled cells into the subject, right after introduction of the labeled cells into the subject, and/or for extended periods after introduction of the labeled cells into the subject (e.g., from weeks to months after introduction of the labeled cells into the subject) in order to track migration of the labeled cells from the site at which they were introduced to and into the target site of interest.

In some embodiments, the subject's own cells are labeled in vivo. For example, iron oxide and/or paramagnetic or superparamagnetic particles can be injected locally, and how cells of interest (e.g., the dendritic cells, macrophages, etc.) take up the iron oxide and/or paramagnetic or superparamagnetic particles can be tested using HP noble gas imaging. In some embodiments, an HP noble gas scan is performed before the injection of the iron oxide and/or the paramagnetic or superparamagnetic particles, and again at a time point after local injection of the iron oxide and/or the paramagnetic or superparamagnetic particles. This permits both identification of where the cells of interest are localized, as well as how the cells of interest take up the iron oxide and/or the paramagnetic or superparamagnetic particles. Additionally, subsequent scans can be employed to track the migration of the labeled cells to and into local lymph nodes. For references related to cell tracking in various organs, see e.g., Butte at al., 2001 and Bulte at al., 2002.

The tracking methods disclosed herein can be employed for determining when and to what extent the cell enters a target site. Additionally, in those embodiments wherein the contrast agent further comprises a therapeutic agent, the tracking methods disclosed herein can be employed for determining when and to what extent the therapeutic agent enters a target site.

The presently disclosed subject matter also provides methods for imaging a cavity in a subject. In some embodiments, the methods comprise (a) administering to the subject a contrast agent comprising a paramagnetic or superparagmanetic material and a targeting moiety that targets the contrast agent to the cavity; (b) introducing into the cavity a hyperpolarized gas; and (c) imaging the cavity by detecting the presence of the paramagnetic or superparagmanetic material on the cavity. In some embodiments, the cavity selected from the group consisting of a sinus, the colon, or the uterus of the subject. In some embodiments, the cavity comprises a target molecule disposed therein, and the targeting moiety binds to the target. In some embodiments, the target molecule is present in or on a cell present in the cavity. In some embodiments, the cell present in the cavity is selected from the group consisting of a cancer cell, optionally a metastasized cancer cell; an inflammatory cell, and a macrophage. In some embodiments, the target molecule is a cytokine. In some embodiments, the paramagnetic or superparagmanetic material comprises a Superparamagnetic Iron Oxide Nanoparticle (SPION).

The presently disclosed subject matter also provides magnetic resonance imaging (MRI) contrast agents comprising a paramagnetic or superparagmanetic material and a targeting moiety, wherein the targeting moiety comprises a ligand that binds to a target present in a tissue to be imaged. In some embodiments, the MRI contrast agent is provided in a carrier or diluent that is pharmaceutically acceptable for use in a human.

The presently disclosed subject matter also provides kits comprising an MRI contrast agent as disclosed herein and a noble gas. In some embodiments, the noble gas is hyperpolarized.

The presently disclosed subject matter also provides systems for magnetic resonance imaging (MRI) comprising a contrast agent. In some embodiments, the contrast agent comprises a paramagnetic or superparagmanetic material, and a targeting moiety, wherein the targeting moiety comprises a ligand that binds to a target present in a tissue to be imaged. In some embodiments, the contrast agent(s) is/are provided in a carrier or diluent that is pharmaceutically acceptable for use in a human. In some embodiments, the presently disclosed systems further comprise a noble gas. In some embodiments, the noble gas is hyperpolarized and/or the system further comprises an apparatus adapted to hyperpolarize the noble gas.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Materials and Methods Employed in the Examples

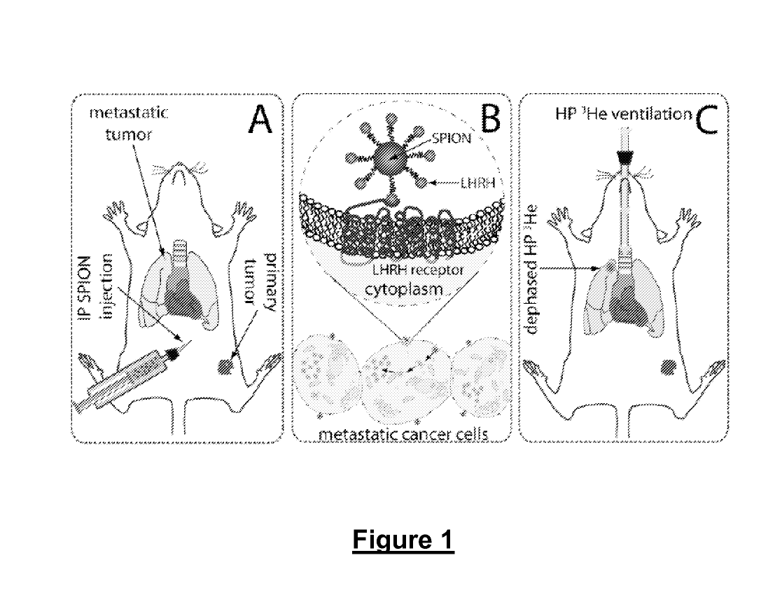

An exemplary approach for visualizing lung metastases using SPIONs in conjunction with hyperpolarized noble gases is depicted in FIG. 1. A mouse with a primary tumor that causes metastatic lesions in the lung is obtained. Optionally, this mouse can be produced by implanting into a site (e.g., the leg, the shoulder, the mammary fat pad, etc.) of the mouse tumor cells of a type that metastasizes to the lung. The tumor cells proliferate at the site to produce the primary tumor, which then metastasizes to the lungs. After sufficient time passed to allow the tumor to metastasize to the lungs, a composition comprising SPIONs is administered to the mouse (e.g., intraperitoneally (i.p.), intravascularly, etc.). The SPIONs travel to the lungs of the mouse, where they contact metastatic cancer cells that are present therein. The SPIONs can bind to the metastatic cancer cells, causing them to be internalized and to collect in the metastatic cancer cells (see FIG. 1B). HP gas (e.g., HP ³He) is administered to the mouse as shown in FIG. 1C, and signal attenuation resulting from the presence of the SPIONs is measured.

Synthesis of SPIONs. Magnetite nanoparticles were synthesized under inert atmospheric conditions with the Schlenk technique as follows: 1.622 g of FeCl₃ and 0.994 g of FeCl₂.4H₂O were placed in a three-necked 100-ml RB flask. To remove even traces of oxygen, the flask was evacuated and flushed three times with nitrogen. The iron salts were dissolved in 25 ml of water, and the solution was stirred magnetically. 2.5 ml of 28% NH₄OH solution was added drop-wise to the solution at room temperature, yielding a black precipitate. This precipitate was heated at 80° C. for 30 minutes, washed several times with water followed by ethanol, and finally dried in a vacuum oven at 70° C.

LHRH Bioconjugation of SPIONs. For the covalent attachment of the LHRH, 60 mg of the magnetite nanoparticles was sonically dispersed in 6 ml of water under nitrogen. A freshly prepared N-Ethyl-N-(3-dimethylaminopropyl)carbodiimide hydrochloride solution (42 mg in 1.5 ml of water) was added, and the total solution was sonicated for 10 more minutes. The mixture was cooled to 4° C., and a solution of 3.7 mg LHRH in 1.5 ml of water was added. The reaction temperature was maintained at 4° C. for 2 hours with occasional swirling of the flask. After 2 hours, the flask was placed on a permanent magnet, and the nanoparticles were allowed to settle. The resulting supernatant was analyzed for unbound LHRH by a quantitative HPLC technique. The LHRH-bound nanoparticles were washed three times with water followed by ethanol, and dried under a slow stream of nitrogen.

³He Polarization. ³He was polarized by a prototype commercial polarizer (IGI.9600.He; Magnetic Imaging Technologies, Durham, N.C., United States of America). The system optically pumped the D₁ electronic transition of rubidium vapor to create a high level of ground state electron spin polarization, which was converted into ³He nuclear polarization through collisions and between the rubidium and helium atoms. The system produced 1.2 L of ³He (Spectra Gases, Alpha, N.J., United States of America) with a final ³He polarization of about 30%. After ³He polarization, the optical cell was cooled to room temperature and the ³He was dispensed into a 150-ml or 300-ml Tedlar bag (Jensen Inert Products, Coral Springs, Fla., United States of America) housed in a Plexiglas cylinder. The cylinder was then attached to a HP gas-compatible ventilator located about 1 m from the opening of a 2T superconducting magnet, where gradient-induced longitudinal ³He relaxation was about 20 minutes (see Driehuys et al., 2007), allowing imaging to be performed with an acceptable signal-to-noise ratio for approximately that duration.

HP ³He MRI. Images were acquired on a 2T superconducting magnet with a horizontal 30-cm clear bore (Oxford Instruments, Oxford, United Kingdom) equipped with 18 G/cm shielded gradients (Resonance Research, Inc, Billerica, Mass., United States of America), controlled by a GE EXCITE® 12.0 console (GE Healthcare, Milwaukee, Wis., United States of America). The scanner was interfaced to the dual-tuned (85.1/64.8 MHz) birdcage coil (5.5 cm by 3.5 cm) using a separate integrated transmit/receive switch with 31-dB gain preamplifier (Nova Medical, Wilmington, Mass., United States of America) for the ¹H or ³He frequency. These frequencies were created by modifying the intrinsic 63.86-MHz frequency of the scanner to either 85.1 MHz for ¹H or 64.8 or MHz for ³He using an up-down converter (Cummings Electronics Labs, North Andover, Mass., United States of America).

The imaging protocol started with a standard ¹H gradient echo MRI to localize the chest cavity. High-resolution 3D HP ³He images were then acquired with a 2 cm field of view (FOV) in the both the coronal and sagittal planes using a 3D radial encoding scheme. Image acquisition used an axial slab excitation to mitigate wrap-around ³He in the trachea. The first, short TE images were acquired with TE/TR=1/5 ms, a constant flip angle of 13°, 20 radial k-space views per breath, and 31.25 kHz bandwidth. Long TE images were acquired using TE/TR=4/8 ms, a flip angle of 17°, and 12 k-space views per breath. After accumulating 20001 radial k-space views, the data were re-gridded on a 128×128×128 Cartesian matrix and Fourier-transformed to give images with a 156×156×156 μm³ Nyquist-limited isotropic resolution.

Animal Care and Use. All experiments were performed according to the guidelines of a protocol approved by the Institutional Animal Care & Use Committee (IACUC) of Duke University (Durham, N.C., United States of America). For these experiments, female and male athymic nude mice (Ncr to nu/nu), 5-6 weeks of age (Charles River Laboratory, Raleigh, N.C., United States of America), were housed in sterile cages and fed autoclaved chow and water ad libitum. Four male mice were inoculated subcutaneously in the flank with 10⁶ Pc-3 human prostate cancer cells suspended in 0.1 ml GFR-MATRIGEL™ (BD Biosciences, San Jose, Calif., United States of America), a basement membrane protein matrix known to improve early angiogenesis. Eight female mice were inoculated with MDA-MB-231 and MDA-MB-435s breast cancer cells, also suspended in GFR-MATRIGEL™. Four mice were inoculated in the mammary fat pads, while the other four were inoculated subcutaneously in the shoulder.

Mice were selected to undergo imaging when the primary tumor reached a size of about 1 cm³. Approximately 24-48 hours prior to imaging, the selected mouse received an i.p. injection using a 27G needle of 100 mg/kg LHRH-SPION suspended in saline. The suspension was prepared using a cell homogenizer to reduce SPION agglomeration and was sonicated for 10 minutes in a bath of ice and water before injection (see e.g., Kumar et al., 2004; Zhou et al., 2006).

Prior to imaging, the animals were anesthetized with an i.p. injection of Nembutal (3 mg/kg of body weight), and anesthesia was maintained during the imaging by repeated i.p. injection of Nembutal, as needed. A tracheotomy was performed to insert a 24-G tracheal tube that extended to 2-3 mm above the carina. The tracheal tube was secured by sutures and connected to a low-dead volume Y-connector separating the inhalation and exhalation lines of an MR-compatible constant volume ventilator. The mouse was ventilated in the supine position with a mixture of 20% oxygen and 80% ³He gas at 100 breaths per minute with a 0.2-ml tidal volume. The airway pressure and ECG of the animal were continuously monitored throughout the study. Body temperature was monitored by a rectal temperature probe and maintained at approximately 37° C. by warm air passing through the magnet bore.

Histology. At the end of the MRI sessions, the animals were sacrificed and the lungs excised, fixed in 10% neutral-buffered formalin, and individually embedded in paraffin blocks. Each paraffin block was then completely sectioned into 200 μm or smaller sections using a microtome and mounted on glass slides. Groups of three adjacent slides were then stained with H&E, Perl Prussian blue, or used for immunohistochemistry (IHC) analysis. Adjacent IHC and H&E slides were then examined under a light microscope to identify and locate small metastases, while Perl Prussian blue staining for iron was used to detect SPION uptake. For IHC analysis, tissue sections were incubated with a mouse monoclonal primary antibody raised against human Gonadotropin-Releasing Hormone Receptor (Clone A9E4, Vector Laboratories, Inc., Burlingame, Calif., United States of America; Catalogue No. VP-G811; GNRHR, also known as the luteinizing hormone releasing hormone receptor (LHRHR) in a 1:20 dilution for 30 minutes. Primary antibody binding was then determined using the Mach 4 Universal Polymer Detection kit (Biocare Medical, Walnut Creek, Calif.; United States of America; Catalogue No. M4U536H) and visualized using Vulcan fast red chromagen.

### Example 1

### Imaging of Metastasized Human Breast Cancer Cells in Mouse Lung

Female BALB/c nude mice at 9 weeks of age were inoculated with MDA-MB-435S human breast cancer cells. These cells metastasized to the lungs and expressed LHRH.

After about 60 days, and 24 hours before the imaging session, the mice received an intraperitoneal (i.p.) injection of 100 mg/kg LHRH-SPIONs (i.e., SPIONs to which LHRH peptides were conjugated). The mice were imaged using a 3D radial acquisition at a resolution of 156×156×1000 μm³. Images were acquired at TE=0.3 ms to show regional ventilation and TE=4 ms for T₂* sensitivity. K-space was filled with 11,520 radial views acquired either 20 per breath with TR/TE=5/0.3 ms or 10 views per breath with TR/TE=10/4 ms. All images used BW=31.25 kHz and a fixed flip angle of 13° or 18°. After imaging, mice were sacrificed and lungs fixed for histology. Histology slides were stained with Prussian Blue to highlight iron so that a correlation could be made between defects seen on the imaging and the location of SPIONs in the lung cancer cells.

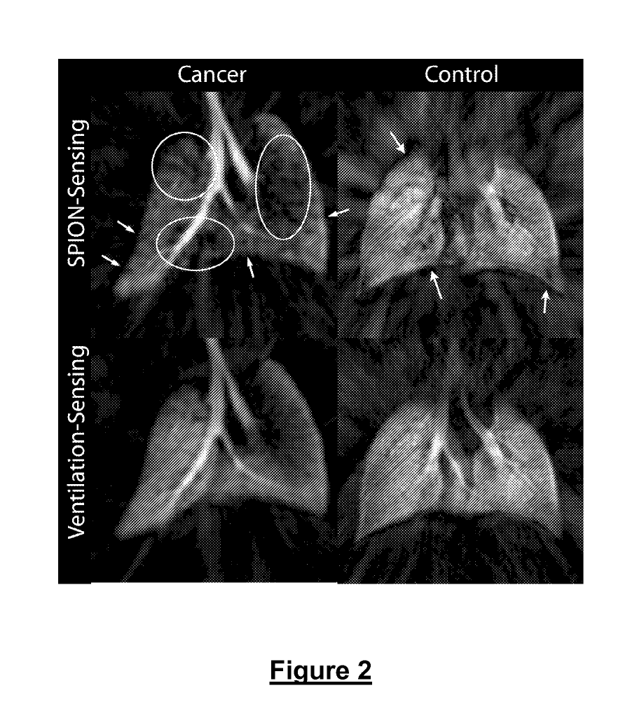

A representative set of images from a tumor-bearing mouse and a control mouse is shown in FIG. 2. Two sets of 3D images were acquired for each mouse as described hereinabove. One was a “SPION-sensing” image (T₂*-weighted with echo time TE=4 ms) and one was a “ventilation-sensing” (spin-density weighted, with TE=0.3 ms). The SPION-sensing image of the tumor-bearing mouse (see top left of FIG. 2) clearly showed numerous signal voids that are highlighted by the circles and arrows. By comparison, the control mouse showed few signal voids. Those that were seen can be attributed to susceptibility effects from nearby blood vessels. Further characterization permits such signal voids due to blood vessels to be better understood and accounted for in order to eliminate potential false positives.

The “ventilation-sensing” scans are shown in the bottom row for the tumor-bearing mouse and the control mouse. The ventilation-sensing scan can ensure that signal voids seen on the SPION-sensing scan are indeed the result of SPION accumulation and not the result of “ventilation defect”. Ventilation defects are areas where ³He did not reach due to some obstruction or impairment of gas flow. In the particular example shown in FIG. 2, ventilation was quite normal. The few areas of darkness on the ventilation scan became even darker, indicating that they were caused by SPIONs.

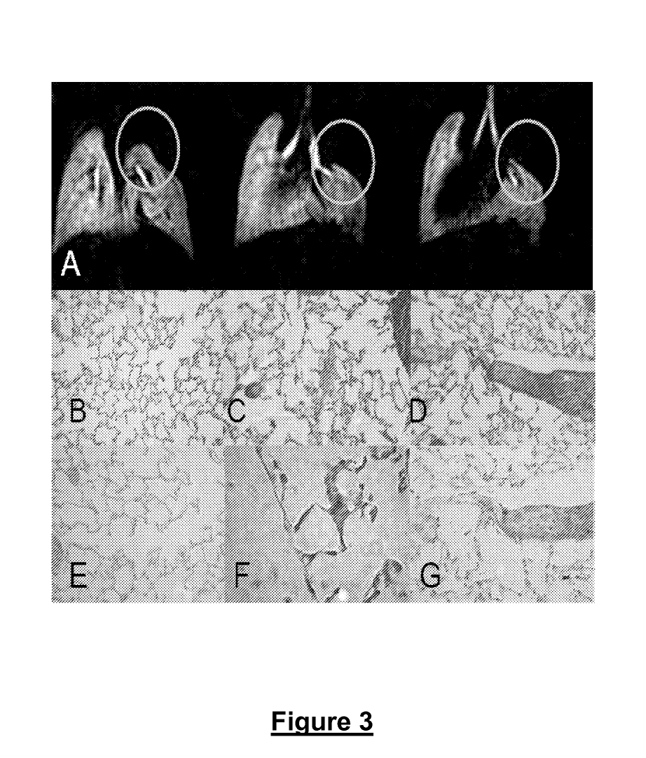

One can also correlate the defects seen on ³He imaging with results of histological assessments. This process was performed in another mouse that was scanned. This mouse had far fewer metastases to the lungs, but nonetheless showed substantial signal voids on the SPION-sensing scan. Regions that showed signal voids were indeed correlated with significant iron accumulation as shown in FIG. 3.

### Example 2

### Imaging of Metastasized Human Prostate Cancer Cells in Mouse Lung

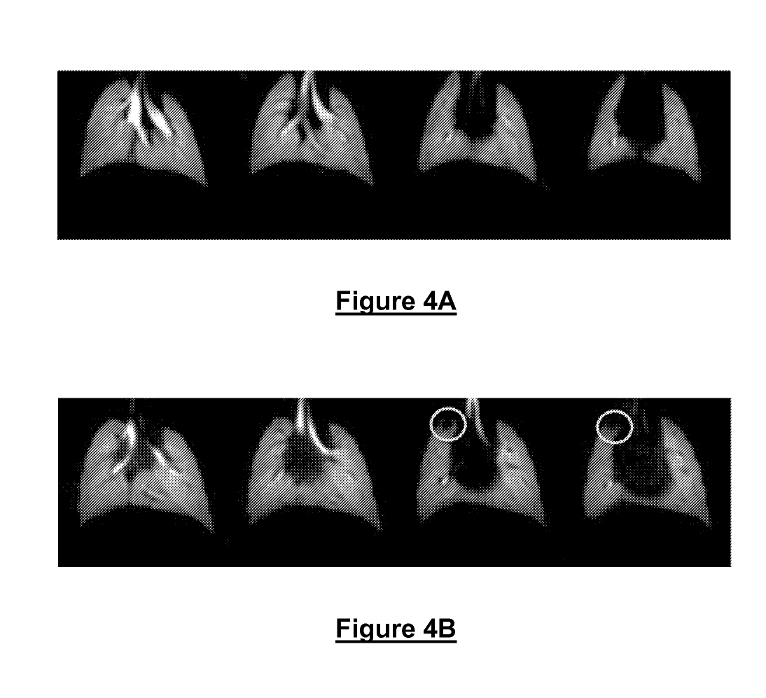

FIG. 4A shows an example of 3D gradient echo ³He images, at relatively long echo time (TE=4 ms), from a control animal that received a SPION injection but no tumor xenograft. FIG. 4B shows similar views of a mouse bearing a primary tumor produced by xenografted human prostate tumor cells. Whereas the control animal showed high signal intensity throughout the lungs, the tumor-bearing animal showed a pronounced signal loss in the right cranial lobe. Such signal voids were representative of all animals studied and were identified in 11 of the 12 tumor-bearing mice. In 10 of the 11 mice, the signal loss was located in the right cranial lobe, while in one mouse, the signal loss affected the entire right lung.

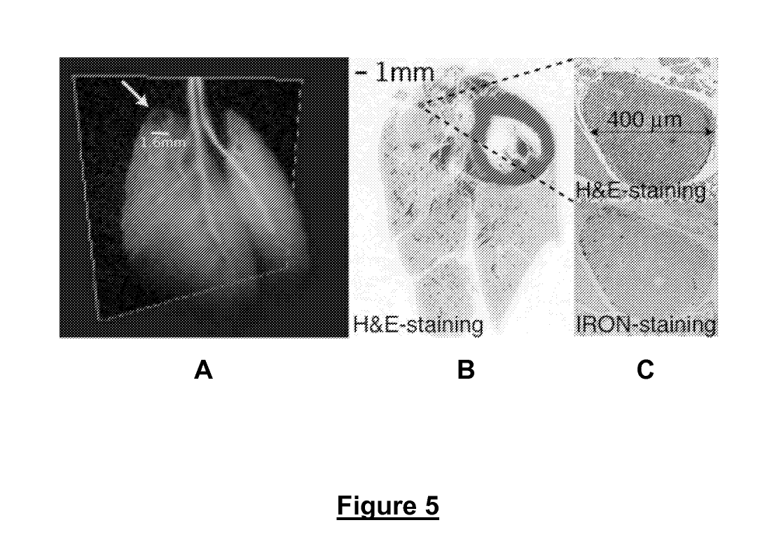

Comparison of ³He MRI against hematoxylin and eosin (H&E) histology (as shown in FIG. 5 for another prostate tumor model mouse) confirmed that ³He signal voids occurred at the site of metastatic tumors that had been, as shown by Prussian-Blue staining, heavily targeted by LHRH-SPIONs. Further immunohistochemical staining confirmed that SPIONs accumulated only in cancer cells with high LHRH receptor expression, while the SPIONs did not accumulate in healthy lung tissue.

Moreover for all mice, the ³He signal void was 4-5 times larger than the actual physical dimensions of the tumor. This observation can be attributed to the “blooming” effect of SPIONs that caused a signal disruption to extend well beyond the targeted cancer cell. The smallest tumor detected on ³He MRI had a size of about 300 μm, while histology confirmed the absence of a tumor in the single mouse that exhibited no signal void in the HP ³He MR images.

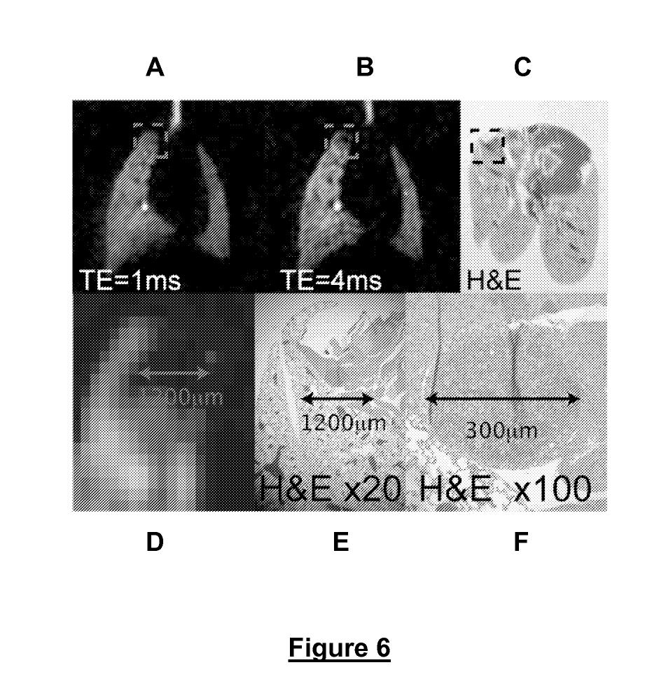

Signal voids in ³He images can also arise from factors other than SPION-targeted cancer cells, like ventilation defects. To distinguish these two effects, the fact that the degree and size of signal attenuation from SPIONs increased with echo time TE whereas ventilation defects were unchanged was exploited. The effects of increasing TE on SPION contrast are shown in FIG. 6, where signal loss, while observable at short echo time (TE=1 ms), was significantly enhanced at longer echo time (TE=4 ms). This pattern was observed in all tumor-bearing mice, indicating that the sensitivity of this technique (i.e., the contrast-to-noise ratio) is enhanced at longer echo times.

### Discussion of Examples 1 and 2

Disclosed herein is the discovery that HP noble gas MRI combined with a targeted SPION contrast agent can detect early-stage metastatic lung tumors in mice. HP noble gas MRI using HP ³He detected 100% of the lesions found by conventional H&E histology. Moreover, all signal voids observed were the result of cancer lesions that had been targeted by SPIONs, while healthy tissue did not show signal defects, thus also suggesting 100% specificity. Although as disclosed herein, metastatic tumors as small as 300 μm in diameter were detected, it is likely that even smaller tumors could be detectable given the high level of image contrast and strong blooming effect from the SPIONs.

The presently disclosed subject matter can also be applied to image SPION accumulation in other body parts that are inaccessible by standard MRI, such as the colon, sinus, uterus, and other cavities. Furthermore, the method could also be used with other hyperpolarized gases (including, but not limited to ³He and/or ¹²⁹Xe).

Also, the combination of labeled magnetic nanoparticles and HP gas MRI is not limited to cancer detection and monitoring, but can also provide a technique for MR imaging of other significant molecular processes in the lungs and other sites. More generally, the method provides the ability to label cells and non-invasively track their fate in the lungs and other sites, a task that can be quite difficult. For example, the basic methodology disclosed herein can allow cancer cell tracking in lungs and other sites to provide critical insights into oncogenic pathways or vulnerability to metastatic invasion, and/or can also permit T cell tracking, which would enhance understanding of T-cell recruitment to, and infiltration into such sites (e.g., within airways in the pathogenesis of asthma). This sensitive and specific imaging method promises to advance diverse areas such as cancer screening, drug development and delivery, and more investigations of parenchymal and airway diseases of the lung as well as of cells, tissues, organs, and other sites of interest.

### Example 3

### Positive Contrast SPION Detection

As disclosed herein, ³He imaging has successfully detected local SPION contrast through darkening of the image (negative contrast). There are also potential advantages to instead image SPIONs using positive contrast.

To this end, He MRI pulse sequences that allow the visualization of viable tumor tissue in mice with human breast or prostate cancer xenografts that have been targeted by LHRH-SPIONs have been developed. These pulse sequences made use of the magnetic field distortion created nearby the SPION particles to allow a background free detection of the targeted area.

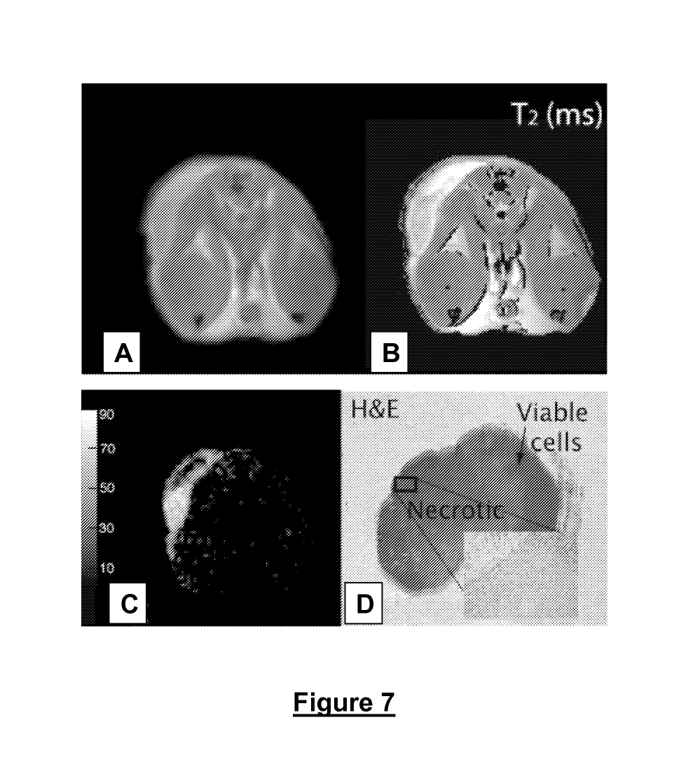

An example of positive contrast SPION detection is presented in FIG. 7, which shows typical images acquired using a conventional spin-echo image, a T2 map, and a positive contrast image. The positive contrast particularly enhanced in the outer area of the primary tumor, later confirmed by histological examination, to be viable and loaded with LHRH-SPIONs.

A methodology using radial gradient echo rather than spin echo imaging, to generate positive contrast for ³He MRI near SPIONs can also be employed.

### REFERENCES

All references listed in the instant disclosure, including but not limited to all patents, patent applications and publications thereof, scientific journal articles, and database entries (including but not limited to GENBANK® database entries and all annotations available therein) are incorporated herein by reference in their entireties to the extent that they supplement, explain, provide a background for, or teach methodology, techniques, and/or compositions employed herein.
Aboud-Pirak et al. (1989) Biochem Pharmacol 38:641-648.
Berry & Curtis (2003) J Phys D: Appl Phys 36:R198-R206.
Branca et al. (2009) Magn Reson Med 61:937-943.
Bulte et al. (2001) Nat Biotechnol 19:1141-1147.
Bulte et al. (2002) Acad Radiol 9 Suppl 2:S332-335.
Chen et al. (1999) Magn Reson Med 42:729-737.
Dillman et al. (1989) Mol Biother 1:250-255.
Driehuys et al. (2007) Magn Reson Med 58:893-900.
Glasspool & Evans (2000) Eur J Cancer 36:1661-1670.
Felfoul et al. (2008) Trans Med Imaging 27:28-35.
FitzGerald & Pastan (1989) J Natl Cancer Inst 81:1455-1463.
Fitzpatrick & Garnett (1995) Anticancer Drug Des 10:1-9.
Freireich et al. (1966) Cancer Chemother Rep 50:219-244.
Harisinghani at al. (2003) N Engl J Med 348:2491-2499 (erratum in (2003) N Engl J. Med 49:1010).
Jemal et al. (2005) CA Cancer J Clin 55:10-30.
Kayton et al. (2006) J Pediatr Surg 41:200-206.
Kim et al. (2007) in: Magjarevic & Nagel (eds.) IFMBE Proceedings. World

Congress on Medical Physics and Biomedical Engineering 2006. 14 (Part 15): 2493-2496.
Kronawitter et al. (1999) Cancer 86:229-235.
Kumar et al. (2004) J Nanosci Nanotechnol 4:245-249.
Lau et al. (1995) Bioorg Med Chem 3:1299-1304.
Leuschner (2006) in: Kumar (ed.) Nanotechnologies for the Life Sciences. Volume 7. Wiley-VCH Verlag GmbH & Co. KGaA, Weinheim, Germany, pages 174-231.
Leuschner et al. (2006) Breast Cancer Res Treat 99:163-176.
Liu at al. (2007) J Neurosci 27:713-722.
Margaritora et al. (2002) Eur J Cardiothorac Surg 21:1111-1114.
Marom & Chasen (2006) Am J Roentgenol 186:289-290.
Marshall & Rabinowitz (1976) J Biol Chem 251:1081-1087.
Muldoon at al. (1995) Am J Pathol 147:1840-1851.
PCT International Patent Application Publication No. WO 98/10795
Reimer et al. (1990) Radiology 177:729-734.
Rohren at al. (2004) Radiology 231:305-332.
Rowland et al. (1993) Cancer Immunol Immunother 37:195-202.
Rubio at al. (2001) Lab Invest 81: 725-734.
Salerno et al. (2001) Eur J Radiol 40:33-44.
Schechter et al. (1991) Int J Cancer 48:167-172.
Seppenwoolde et al. (2007) Magn Reson Med 58:605-609.
Shawler et al. (1988) J Biol Response Mod 7:608-618.
Shih et al. (1994) Cancer Immunol Immunother 38:92-98.
Silvestri et al. (2003) Chest 123(1 suppl):147S-156S.
Sivam et al. (1995) Cancer Res 55:2352-2356.
U.S. Patent Application Publication No. 20060140871.
U.S. Pat. Nos. 5,545,396; 5,612,103; 5,642,625; 5,773,413; 5,785,953; 5,789,921; 5,809,801; 5,860,295; 5,861,478; 6,079,213; 6,123,919; 6,128,918; 6,180,082; 6,199,385; 6,237,363; 6,241,966; 6,284,222; 6,295,834; 6,305,190; 6,346,229; 6,427,452; 6,430,960; 6,484,532; 6,488,910; 6,491,895; 6,526,778; 6,537,825; 6,593,144; 6,599,497; 6,630,126; 6,667,008; 6,696,040; 6,875,744; 6,991,777; and 7,381,704.
Wang et al. (2001) Eur J Radiol 11:2319-2331.
Ward et al. (2000) J Magn Reson 143:79-87.
Walther & Stein (1999) Mol Biotechnol 13:21-28.
Zhou et al. (2006) Biomaterials 27:2001-2008.
Zurkiya & Hu (2006) Magn Reson Med 56:726-732.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for imaging a target cell, tissue, or organ in a subject, the method comprising:
(a) administering to the subject a contrast agent comprising a paramagnetic or superparagmanetic material and a targeting moiety that targets the contrast agent to the cell, tissue, or organ;
(b) introducing into the target cell, tissue, or organ, or into the vicinity thereof, a hyperpolarized gas; and
(c) imaging the target cell, tissue, or organ by detecting the presence of the contrast agent in, on, and/or near the target cell, tissue, or organ.

2. The method of claim 1, wherein the paramagnetic or superparagmanetic material comprises a Superparamagnetic Iron Oxide Nanoparticle (SPION).

3. The method of claim 1, wherein the target cell, tissue, or organ comprises a cancer cell.

4. The method of claim 3, wherein the cancer cell is a metastasized cancer cell.

5. The method of claim 4, wherein the metastasized cancer cell is present in the lung of the subject.

6. The method of claim 1, wherein the target cell, tissue, or organ comprises a target, and the targeting moiety comprises a molecule that binds to the target.

7. The method of claim 6, wherein the molecule that binds to the target comprises a peptide, an antibody or a fragment or derivative thereof, or a small molecule.

8. The method of claim 7, wherein the antibody or the fragment or derivative thereof comprises a paratope that binds to the target.

9. The method of claim 7, wherein the peptide comprises a ligand or receptor that binds to a receptor or ligand present on the surface of the cell or on the surface of a cell present in the tissue or organ.

10. The method of claim 9, wherein the ligand comprises a luteinizing hormone releasing hormone (LHRH) peptide or a fragment or derivative thereof that binds to a luteinizing hormone releasing hormone receptor present on the target cell or in a cell present in the target tissue or organ.

11. The method of claim 1, wherein the hyperpolarized gas comprises ³He, ¹²⁹Xe, or a combination thereof.

12. The method of claim 11, wherein the hyperpolarized gas comprises ¹²⁹Xe.

13. The method of claim 1, wherein the imaging comprises a contrast agent-sensing scan with an echo time of from about 3 to about 12 ms and a ventilation-sensing scan with echo time of from about 0.3 to about 1 ms.

14. The method of claim 13, wherein the contrast agent-sensing scan has an echo time of about 4 ms.

15. The method of claim 13, wherein the ventilation-sensing sensing scan comprises an echo time of about 0.3 ms.

16. The method of claim 1, wherein the imaging is based on under-sampled radial imaging.

17. The method of claim 1, wherein the imaging is based on Cartesian sampling.

18. The method of claim 1, wherein the target cell comprises a cancer cell present in or surrounding a gas-accessible cavity selected from the group consisting of a sinus, the colon, and the uterus of the subject.

19. A method for screening for metastasis of a tumor and/or a cancer to the lung of a subject, the method comprising:
(a) administering to the subject a contrast agent comprising a paramagnetic or superparagmanetic material and a targeting moiety that binds to a tumor and/or a cancer cell;
(b) introducing a hyperpolarized gas into the lung of the subject; and
(c) imaging the lung by detecting the presence of the contrast agent bound to the tumor and/or the cancer cell in the lung.

20. The method of claim 19, wherein the tumor and/or the cancer comprises a tumor and/or a cancer of the breast, prostate, uterus, or lung.

21. A method for imaging a target cell, tissue, or organ in and/or adjacent to a cavity in a subject, the method comprising:
(a) administering to the subject a contrast agent comprising a paramagnetic or superparagmanetic material and a targeting moiety that targets the contrast agent to the cell, tissue, or organ;
(b) introducing into the cavity a hyperpolarized gas; and
(c) imaging the target cell, tissue, or organ by detecting the presence of the contrast agent in or on the target cell, tissue, or organ.

22. The method of claim 21, wherein the paramagnetic or superparagmanetic material comprises a Superparamagnetic Iron Oxide Nanoparticle (SPION).

23. The method of claim 21, wherein the target cell, tissue, or organ comprises a cancer cell.

24. The method of claim 21, wherein the target cell, tissue, or organ comprises a target, and the targeting moiety comprises a molecule that binds to the target.

25. The method of claim 24, wherein the molecule that binds to the target comprises a peptide, an antibody or a fragment or derivative thereof, or a small molecule.

26. The method of claim 25, wherein the antibody or the fragment or derivative thereof comprises a paratope that binds to the target.

27. The method of claim 26, wherein the peptide comprises a ligand or receptor that binds to a receptor or ligand present on the surface of the cell or on the surface of a cell present in the tissue or organ.

28. The method of claim 21, wherein the hyperpolarized gas comprises ³He, ¹²⁹Xe, or a combination thereof.

29. The method of claim 28, wherein the hyperpolarized gas comprises ¹²⁹Xe.

30. The method of claim 21, wherein the imaging comprises a contrast agent-sensing scan with an echo time of from about 3 to about 12 ms and a ventilation-sensing scan with echo time of from about 0.3 to about 1 ms.

31. The method of claim 30, wherein the contrast agent-sensing scan has an echo time of about 4 ms.

32. The method of claim 30, wherein the ventilation-sensing sensing scan comprises an echo time of about 0.3 ms.

33. The method of claim 21, wherein the imaging is based on under-sampled radial imaging.

34. The method of claim 21, wherein the imaging is based on Cartesian sampling.

35. The method of claim 21, wherein the cavity selected from the group consisting of a sinus, the colon, or the uterus of the subject.

36. A method for imaging a cavity in a subject, the method comprising:
(a) administering to the subject a contrast agent comprising a paramagnetic or superparagmanetic material and a targeting moiety that targets the contrast agent to the cavity;
(b) introducing into the cavity a hyperpolarized gas; and
(c) imaging the cavity by detecting the presence of the contrast agent on the cavity.

37. The method of claim 36, wherein the cavity selected from the group consisting of a sinus, the colon, or the uterus of the subject.

38. The method of claim 36, wherein the cavity comprises a target molecule disposed therein, and the targeting moiety binds to the target.

39. The method of claim 36, wherein the target molecule is present in or on a cell present in the cavity.

40. The method of claim 39, wherein the cell present in the cavity is selected from the group consisting of a cancer cell, optionally a metastasized cancer cell, an inflammatory cell, and a macrophage.

41. The method of claim 36, wherein the target molecule is a cytokine.

42. The method of claim 36, wherein the paramagnetic or superparagmanetic material comprises a Superparamagnetic Iron Oxide Nanoparticle (SPION).

43. The method of any of claims 1, 19, 21, and 36, wherein the detecting the presence of the contrast agent comprises detecting a signal void that results from the presence of the contrast agent.

44. The method of any of claims 1, 19, 21, and 36, wherein the method further comprises administering the hyperpolarized gas and pre-imaging the cell, tissue, and/or organ before administering the contrast agent.

45. The method of any of claims 1, 19, 21, and 36, wherein the contrast agent further comprises a therapeutic agent.

46. The method of claim 45, wherein the therapeutic agent is selected from the group consisting of a cytotoxic agent, a chemotherapeutic agent, a radionuclide, an antimetabolite, a suicide gene product, a toxin, a lytic peptide, and combinations thereof.

47. A method for tracking a cell in a subject, the method comprising:
(a) labeling a cell with iron oxide, a paramagnetic or superparamagnetic material, or both, wherein the labeling is in vitro, in vivo, or both;
(b) administering the cell to the subject if the cell was labeled in vitro; and
(c) serially imaging one or more sites in the subject with hyperpolarized noble gas cell, wherein the imaging is performed before the introduction of the labeled cell into the subject, right after introduction of the labeled cell into the subject, and/or for extended periods after introduction of the labeled cell into the subject
whereby the cell is tracked in the subject.

48. The method of claim 47, wherein the cell is a stem cell, a T cell, a macrophage, a dendritic cell, or a cancer cell.

49. The method of claim 47, wherein the cell is labeled in vivo by introducing into the subject the iron oxide, the paramagnetic or superparamagnetic material, or both in a site in which the iron oxide, the paramagnetic or superparamagnetic material, or both come in contact with the cell to thereby label the cell.

50. The method of claim 47, wherein the cell is tracked to a target site selected from the group consisting of a lymph node and a lung.

51. A method for MRI imaging an embolism in a subject, the method comprising:
(a) administering to the subject a hyperpolarized gas; and
(b) imaging the embolism in the subject by detecting an attenuating effect on an MRI signal generated by the embolism.

52. A magnetic resonance imaging (MRI) contrast agent comprising a paramagnetic or superparagmanetic material and a targeting moiety, wherein the targeting moiety comprises a ligand that binds to a target present on a cell and/or in a tissue to be imaged.

53. The MRI contrast agent of claim 52, provided in a carrier or diluent that is pharmaceutically acceptable for use in a human.

54. A kit comprising the MRI contrast agent of claim 52 and a noble gas.

55. The kit of claim 54, wherein the noble gas is hyperpolarized.

56. A system for magnetic resonance imaging (MRI) comprising a contrast agent, wherein the contrast agent comprises a paramagnetic or superparagmanetic material, and a targeting moiety, wherein the targeting moiety comprises a ligand that binds to a target present in a tissue to be imaged.

57. The system of claim 56, wherein the contrast agent is provided in a carrier or diluent that is pharmaceutically acceptable for use in a human.

58. The system of claim 56, further comprising a noble gas.

59. The system of claim 58, wherein the noble gas is hyperpolarized, or wherein the system further comprises an apparatus adapted to hyperpolarize the noble gas.
